(19) United States
(12) Patent Application Publication
(10) Pub. No.: US 2025/0127425 A1
(43) Pub. Date: Apr. 24, 2025

(54) GAIT MEASUREMENT DEVICE, GAIT MEASUREMENT SYSTEM, GAIT MEASUREMENT METHOD, AND RECORDING MEDIUM

(71) Applicant: NEC Corporation, Minato-ku, Tokyo (JP)

(72) Inventor: Hiroshi Kajitani, Tokyo (JP)

(73) Assignee: NEC Corporation, Minato-ku, Tokyo (JP)

(21) Appl. No.: 18/691,493

(22) PCT Filed: Oct. 11, 2021
(86) PCT No.: PCT/JP2021/037516

§ 371 Date: Mar. 13, 2024

(51) Int. Cl. A61B 5/11

(52) U.S. Cl. CPC A61B 5/112; A61B 2562/0219

(57) ABSTRACT

Provided is a gait measurement device that includes an acquisition unit that acquires sensor data related to a motion of a foot, an interpolation unit that interpolates interpolation data into a period in which the sensor data is lost, a calculation unit that calculates a gait parameter by using the sensor data obtained by interpolating the interpolation data by the interpolation unit, and a transmission unit that transmits the gait parameter calculated by the calculation unit.

### TECHNICAL FIELD

The present disclosure relates to a gait measurement device or the like that measures a gait by using sensor data related to a motion of a foot.

### BACKGROUND ART

In response to an increase in interest in healthcare, attention has been focused on services for providing users with information based on features (also referred to as “gait”) included in a walking pattern. For example, a technique of analyzing a gait of a user by using sensor data measured by a sensor mounted on footwear such as shoes has been developed. The data measured by the sensor is transmitted to a mobile terminal carried by the user via wireless communication such as Bluetooth (registered trademark). In order to implement the gait measurement in real time, it is desirable that the data measured by the sensor is transmitted to the mobile terminal at an appropriate timing.

PTL 1 discloses a walking posture meter that presents a temporal transition of a quality of a walking posture in daily life to a user. The walking posture meter disclosed in PTL 1 includes an acceleration sensor, an evaluation unit, and a display processing unit. The acceleration sensor is mounted on a median line of a waist of a subject. The evaluation unit repeatedly obtains an evaluation amount quantitatively representing the walking posture of the subject based on an output of the acceleration sensor at intervals of predetermined unit periods within a predetermined consecutive gait period of equal to or less than 10 minutes. The display processing unit displays the repeatedly obtained evaluation amounts on the display screen side by side in chronological order.

### CITATION LIST

### Patent Literature

PTL 1: JP 2014-217694 A

### SUMMARY OF INVENTION

### Technical Problem

In the technique disclosed in PTL 1, the output of the acceleration sensor mounted on the waist of the subject is processed by the control unit. In the technique disclosed in PTL 1, the control unit operating as the evaluation unit acquires an output of accelerations in a logging period included in a plurality of unit periods in the consecutive gait period. On the other hand, the control unit does not acquire an output of accelerations in a period (non-logging period) other than the logging period included in the unit periods. Therefore, in the technique disclosed in PTL 1 the acceleration data in the non-logging period is lost. If the gait parameter is calculated in a state in which the acceleration data in the non-logging period is lost, the influence of the loss of the acceleration data becomes more significant as the number of steps increases, leading to a decrease in gait measurement accuracy.

It is an object of the present disclosure to provide a gait measurement device or the like which is capable of interpolating the loss of the sensor data and performing highly accurate gait measurement.

### Solution to Problem

A gait measurement device according to an aspect of the present disclosure includes an acquisition unit that acquires sensor data related to a motion of a foot, an interpolation unit that interpolates interpolation data into a period in which the sensor data is lost, a calculation unit that calculates a gait parameter by using the sensor data obtained by interpolating the interpolation data by the interpolation unit, and a transmission unit that transmits the gait parameter calculated by the calculation unit.

A gait measurement method performed by a computer includes acquiring sensor data regarding a motion of a foot, interpolating interpolation data into a period in which the sensor data is lost, calculating a gait parameter by using the sensor data obtained by interpolating the interpolation data, and transmitting the calculated gait parameter.

A non-transitory recording medium according to an aspect of the present disclosure stores a program causing a computer to execute a process of acquiring sensor data regarding a motion of a foot, a process of interpolating interpolation data into a period in which the sensor data is lost, a process of calculating a gait parameter by using the sensor data obtained by interpolating the interpolation data, and a process of transmitting the calculated gait parameter.

### Advantageous Effects of Invention

According to the present disclosure, it is possible to provide a gait measurement device or the like which is capable of interpolating the loss of the sensor data and performing highly accurate gait measurement.

### BRIEF DESCRIPTION OF DRAWINGS

FIG. 1 is a block diagram illustrating an example of a configuration of a gait measurement device according to a first example embodiment.

FIG. 2 is a conceptual diagram illustrating an arrangement example of the gait measurement device according to the first example embodiment.

FIG. 3 is a conceptual diagram for describing a coordinate system set in the gait measurement device according to the first example embodiment.

FIG. 4 is a conceptual diagram for describing human body planes serving as a reference of sensor data measured by the gait measurement device according to the first example embodiment.

FIG. 5 is a conceptual diagram for describing a sole angle measured by the gait measurement device according to the first example embodiment.

FIG. 6 is a conceptual diagram for describing a walking event detected by the gait measurement device according to the first example embodiment.

FIG. 7 is a conceptual diagram for describing an example of a walking waveform of a roll angle measured by the gait measurement device according to the first example embodiment.

FIG. 8 is a conceptual diagram for describing an example of a walking waveform of acceleration in a traveling direction measured by the gait measurement device according to the first example embodiment.

FIG. 9 is a conceptual diagram for describing an example of a walking waveform (with no loss) measured by the gait measurement device according to the first example embodiment.

FIG. 10 is a conceptual diagram for describing an example of a walking waveform (with loss) measured by the gait measurement device according to the first example embodiment.

FIG. 11 is a conceptual diagram for describing an example of data interpolation by the gait measurement device according to the first example embodiment.

FIG. 12 is a block diagram illustrating an example of a detailed configuration of a gait measurement device according to the first example embodiment.

FIG. 13 is a flowchart for describing an example of an operation of the gait measurement device according to the first example embodiment.

FIG. 14 is a flowchart for describing an example of a sensor data measurement process by the gait measurement device according to the first example embodiment.

FIG. 15 is a flowchart for describing an example of a gait parameter calculation process by the gait measurement device according to the first example embodiment.

FIG. 16 is a block diagram illustrating an example of a configuration of a gait measurement system according to a second example embodiment.

FIG. 17 is a conceptual diagram illustrating an example in which information related to a body condition of a user output by the gait measurement system according to the second example embodiment is displayed on a screen of a mobile terminal.

FIG. 18 is a block diagram illustrating an example of a configuration of a gait measurement device according to a third example embodiment.

FIG. 19 is a block diagram illustrating an example of a hardware configuration that executes control and a process according to each example embodiment.

### EXAMPLE EMBODIMENTS

Hereinafter, example embodiments of the present invention will be described with reference to the drawings. However, the example embodiments described below have technically preferable limitations for carrying out the present invention, but the scope of the invention is not limited to the following description. In all the drawings used in the following description of the example embodiment, the same reference numerals are given to the same parts unless there is a particular reason. Further, in the following example embodiments, repeated description of similar configurations and operations may be omitted.

### First Example Embodiment

First, a measurement device according to a first example embodiment will be described with reference to the drawings. The measurement device of the present example embodiment measures features (also referred to as “gait”) included in the walking pattern of the user by using sensor data measured in response to the walking of the user. The measurement device according to the present example embodiment interpolates a loss of sensor data that has not been acquired in a communication period or the like. Hereinafter, a system in which the right foot is a reference foot and the left foot is an opposite foot will be described. The technique of the present example embodiment can also be applied to a system in which the left foot is a reference foot and the right foot is an opposite foot.

### (Configuration)

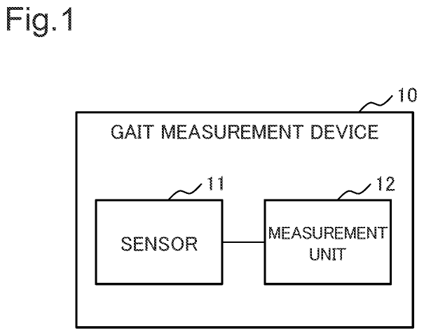

FIG. 1 is a block diagram illustrating a configuration of a gait measurement device 10 of the present example embodiment. The gait measurement device 10 includes a sensor 11 and a measurement unit 12. The sensor 11 and the measurement unit 12 are configured with a single package. For example, the sensor 11 and the measurement unit 12 may be configured with individual packages. For example, the sensor 11 may be removed from the configuration of the gait measurement device 10, and the gait measurement device 10 may be configured only with the measurement unit 12. The gait measurement device 10 is installed on a foot portion. For example, the gait measurement device 10 is installed on footwear such as shoes. Hereinafter, the description will proceed with an example in which the gait measurement device 10 is arranged at a position on the back side of the arch of the foot.

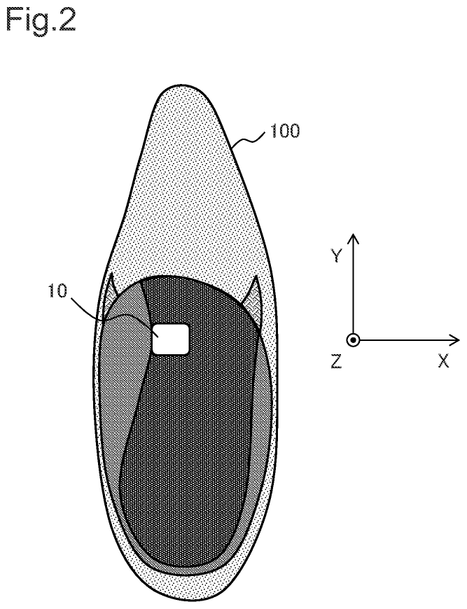

FIG. 2 is a conceptual diagram illustrating an example in which the gait measurement device 10 is arranged in a shoe 100. In the example of FIG. 2, the gait measurement device 10 is installed at a position associated with the back side of the arch of the foot. For example, the gait measurement device 10 is arranged in an insole inserted into the shoe 100. For example, the gait measurement device 10 is arranged on the bottom surface of the shoe 100. For example, the sensor 11 is embedded in the main body of the shoe 100. The gait measurement device 10 may be detachable from the shoe 100 or may not be detachable from the shoe 100. The gait measurement device 10 may be installed at a position other than the back side of the arch of the foot as long as the sensor data related to the motion of the foot can be acquired. The gait measurement device 10 may be installed on a sock worn by the user or a decorative article such as an anklet worn by the user. The gait measurement device 10 may be directly attached to the foot or may be embedded in the foot. FIG. 2 illustrates an example in which the gait measurement device 10 is installed in the shoe 100 on the right foot side. The gait measurement device 10 may be installed on the shoe 100 on the left foot side. The gait measurement device 10 may be installed on the shoes 100 of both feet. If the gait measurement device 10 is installed in the shoes 100 of both legs/feet, the body condition can be estimated based on the motions of both legs/feet.

The sensor 11 includes an acceleration sensor and an angular velocity sensor. The sensor 11 measures, as the physical quantity related to the motion of the foot of the user wearing the footwear, a physical quantity such as an acceleration (also referred to as a “spatial acceleration”) measured by the acceleration sensor or an angular velocity (also referred to as a “spatial angular velocity”) measured by the angular velocity sensor. The physical quantity related to the motion of the foot measured by the sensor 11 also includes a speed, an angle, or a position (loci) calculated by performing integral on acceleration or angular velocity. The sensor 11 converts the measured physical quantity into digital data (also referred to as “sensor data”). The sensor 11 outputs the converted sensor data to the measurement unit 12.

The sensor 11 is implemented by, for example, an inertial measurement device including an acceleration sensor and an angular velocity sensor. An example of the inertial measurement device is an inertial measurement unit (IMU). The IMU includes an acceleration sensor that measures accelerations in three axial directions and an angular velocity sensor that measures angular velocities around the three axes. The sensor 11 may be implemented by an inertial measurement device such as a vertical gyro (VG) or an attitude heading (AHRS). The sensor 11 may be implemented by global positioning system/inertial navigation system (GPS/INS). The sensor 11 is not limited to the inertial measurement device as long as it can measure the physical quantity related to the motion of the foot.

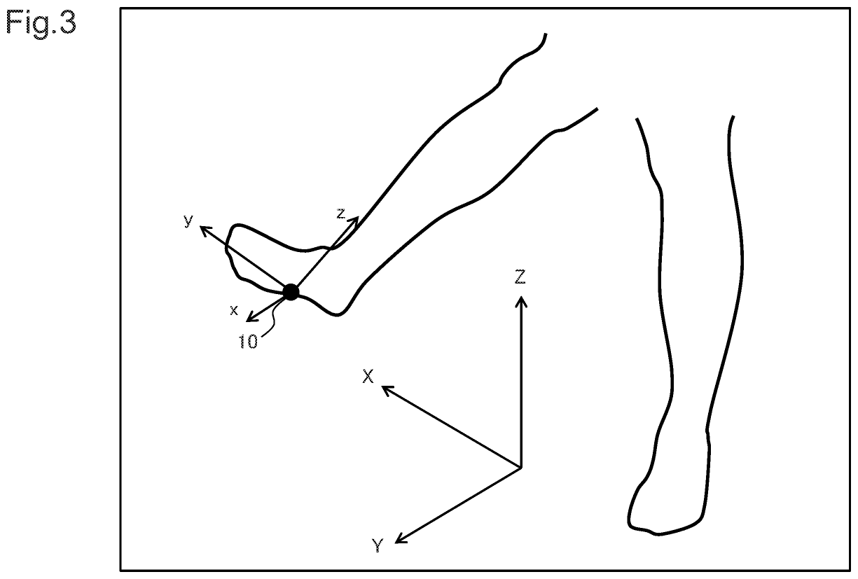

FIG. 3 is a conceptual diagram for describing a local coordinate system (an x axis, a y axis, and a z axis) set in the gait measurement device 10 and a world coordinate system (an X axis, a Y axis, and a Z axis) set with respect to the ground surface in a case in which the gait measurement device 10 is installed on the back side of the arch of the foot. In the world coordinate system (the X axis, the Y axis, and the Z axis), in a state in which the user is standing upright, a lateral direction of the user is set as an X-axis direction (right side is positive), a front direction of the user (traveling direction) is set as a Y-axis direction (front side is positive), and a gravity direction is set as a Z-axis direction (vertically upper side is positive). In the present example embodiment, the local coordinate system including the x direction, the y direction, and the z direction with reference to the gait measurement device 10 is set. The local coordinate system set in the gait measurement device 10 is not limited to the example of FIG. 3. A specific local coordinate system can be set for the gait measurement device 10.

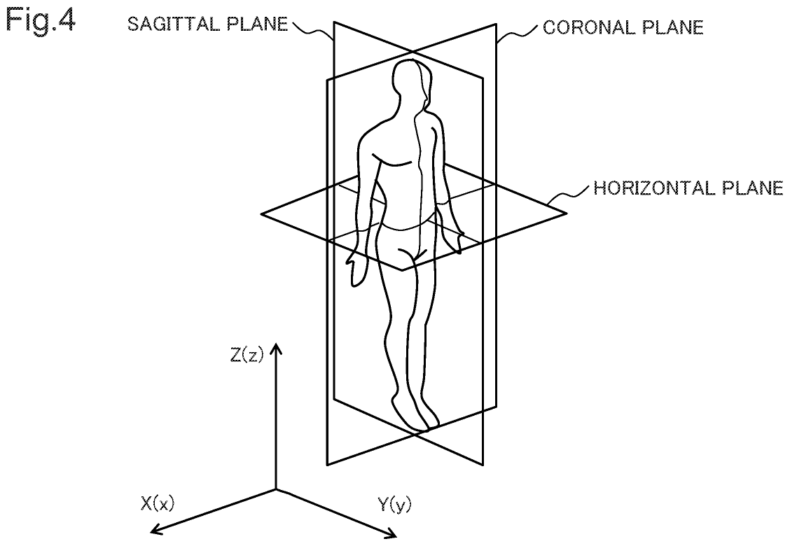

FIG. 4 is a conceptual diagram for describing planes (also referred to as “human body planes”) set for the human body. In the present example embodiment, a sagittal plane dividing the body into left and right, a coronal plane dividing the body into front and rear, and a horizontal plane dividing the body horizontally are defined. In the example of FIG. 4, it is assumed that the world coordinate system and the local coordinate system coincide with each other in an upright state. In the present example embodiment, rotation of the sagittal plane with the x axis as the rotation axis is defined as a roll, rotation of the coronal plane with the y axis as the rotation axis is defined as a pitch, and rotation of the horizontal plane with the z axis as the rotation axis is defined as a yaw. A rotation angle of the sagittal plane with the x axis as the rotation axis is defined as a roll angle, the rotation angle of the coronal plane with the y axis as the rotation axis is defined as a pitch angle, and the rotation angle of the horizontal plane with the z axis as a rotation axis is defined as a yaw angle.

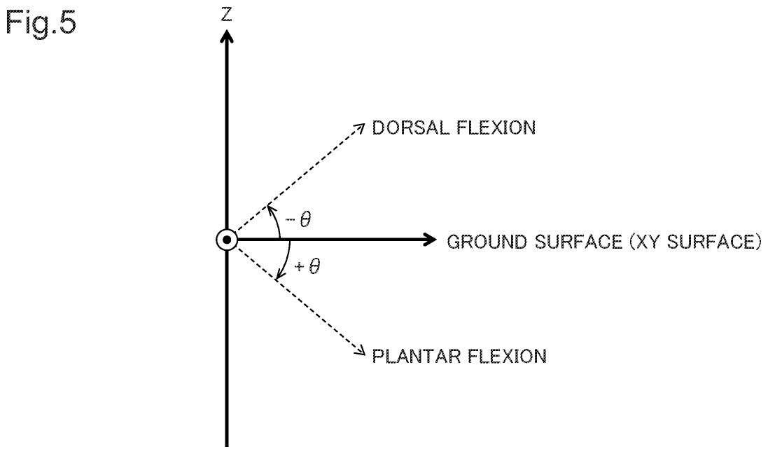

FIG. 5 is a conceptual diagram for describing a sole angle (roll angle). The sole angle is an angle of the sole relative to the ground surface (an XY plane). The sole angle is also called a posture angle. In the present example embodiment, whether the sole angle is positive or negative is defined in such a way that a state in which the toe is located above the heel (dorsal flexion) is negative, and a state in which the toe is located below the heel (plantar flexion) is positive.

The measurement unit 12 (also referred to as a measurement device) acquires the sensor data measured in response to walking of the user from the sensor 11. The measurement unit 12 generates time-series data (also referred to as a walking waveform) of the acquired sensor data. For example, the measurement unit 12 generates a walking waveform related to an acceleration or a speed in the three axial directions, a position (loci), or an angular velocity or an angle around the three axes. Here, the walking waveform is not the time-series data of the sensor data which is represented as a graph, but is the time-series data of the sensor data itself.

For example, the measurement unit 12 is implemented by a microcomputer or a microcontroller. For example, the measurement unit 12 includes a control circuit and a storage circuit. For example, the control circuit is implemented by a central processing unit (CPU). For example, the storage circuit is implemented by a volatile memory such as a random access memory (RAM). For example, the storage circuit is implemented by a non-volatile memory such as a read only memory (ROM) or an electrically erasable and programmable read only memory (EEPROM).

The measurement unit 12 acquires the angular velocity and the acceleration measured by the acceleration sensor 111 and the angular velocity sensor 112. For example, the measurement unit 12 performs analog-to-digital conversion (AD conversion) on the acquired physical quantities (analog data) such as the angular velocity and the acceleration, and stores the converted digital data in the EEPROM. The physical quantity (analog data) measured by the acceleration sensor 111 and the angular velocity sensor 112 may be converted into the digital data in each of the acceleration sensor 111 and the angular velocity sensor 112. The digital data stored in the EEPROM is transmitted at a predetermined timing.

The measurement unit 12 detects a predetermined walking event from the generated walking waveform based on the feature appeared in the walking waveform. For example, the measurement unit 12 detects a timing of a characteristic change associated with the appearance of the walking event in the walking waveform. For example, the measurement unit 12 detects a characteristic maximum or minimum timing associated with the appearance of the walking event in the walking waveform.

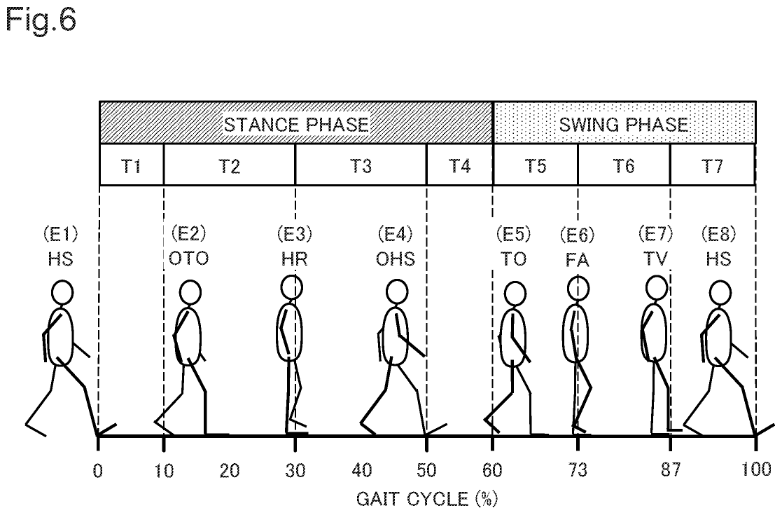

FIG. 6 is a conceptual diagram for describing a walking event detected in one walking cycle with the right foot as a reference. A horizontal axis of FIG. 6 indicates a walking cycle in which one walking cycle of the right foot is normalized as 100% (%), and a point of time at which the heel of the right foot touches the ground surface is a start point, and a point of time at which the heel of the right foot next touches the ground is an end point. One walking cycle of one foot is roughly divided into a stance phase in which at least a part of the back side of the foot is in contact with the ground surface and a swing phase in which the back side of the foot is separated from the ground surface. In the example of FIG. 6, normalization is performed in such a way that the stance phase occupies 60%, and the swing phase occupies 40%. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. In the walking waveform of one walking cycle, the point of time point at which the heel touches the ground surface may not be used as the start point. For example, the start point of the walking waveform of one walking cycle may be set to a point of time in the middle of the stance phase.

In FIG. 6, a walking event E1 indicates an event (heel strike) in which the heel of the right foot touches the ground surface (HS: Heel Strike). A walking event E2 indicates an event (opposite toe off) in which the toe of the left foot is separated from the ground surface in a state in which a contact surface of the sole of the right foot is in contact with the ground surface (OTO: Opposite Toe Off). A walking event E3 indicates an event (heel rise) in which the heel of the right foot lifts in a state in which the contact surface of the sole of the right foot is in contact with the ground surface (HR: Heel Rise). A walking event E4 is an event (opposite heel strike) in which the heel of the left foot touches the ground surface (OHS: Opposite Heel Strike). A walking event E5 indicates an event (toe off) in which the toe of the right foot is separated from the ground surface in a state in which the contact surface of the sole of the left foot is in contact with the ground surface (TO: Toe Off). A walking event E6 indicates an event (foot adjacent) in which the left foot and the right foot cross each other in a state in which the contact surface of the sole of the left foot is in contact with the ground surface (FA: Foot Adjacent). A walking event E7 indicates an event (tibia vertical) in which the tibia of the right foot is approximately perpendicular to the ground surface in a state in which the sole of the left foot is in contact with the ground surface (TV: Tibia Vertical). A walking event E8 indicates an event (heel strike) in which the heel of the right foot touches the ground surface (HS: Heel Strike). The walking event E8 is regarded as the end point of the walking cycle starting from the walking event E1 and regarded as the start point of the next walking cycle.

For example, the measurement unit 12 detects the toe off or the heel strike as a predetermined walking event. In a state in which the toe is located below the heel (plantar flexion), the roll angle becomes maximum at the timing of the toe off. For example, the measurement unit 12 detects, as the timing of the toe off, a timing at which the roll angle becomes maximum in the walking waveform of one walking cycle. In a state in which the toe is located above the heel (dorsal flexion), the roll angle becomes minimum at the timing of the heel strike. For example, the measurement unit 12 detects, as the timing of the heel strike, a timing at which the roll angle becomes minimum in the walking waveform of one walking cycle.

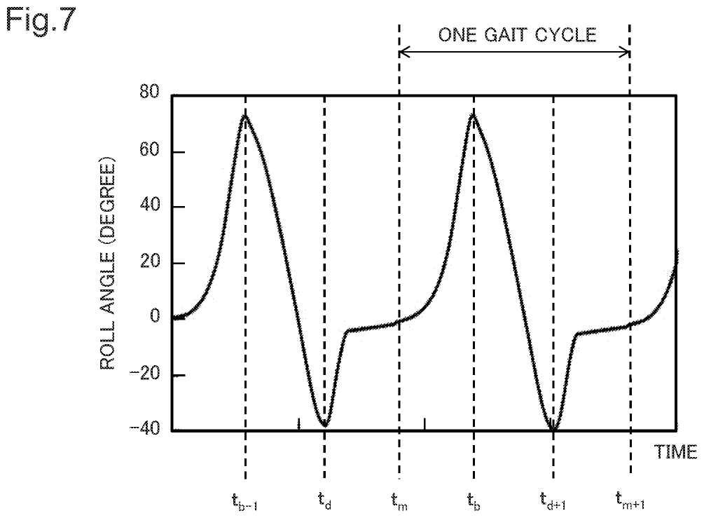

For example, the measurement unit 12 detects, as the predetermined walking event, a timing in the middle of the stance phase from the walking waveform of the roll angle. FIG. 7 is a graph of an example of the walking waveform (roll angle) for one walking cycle. A time ta at which the walking waveform becomes minimum is a timing of the start of the stance phase (heel strike). A time t_{b} at which the walking waveform becomes maximum is a timing of the start of the swing phase (toe off). A time of a midpoint between the time t_{d} of the start of the stance phase and the time t_{b} of the start of the swing phase is the timing in the middle of the stance phase. The measurement unit 12 sets the time of the timing in the middle of the stance phase as the time of the start point of one walking cycle (also referred to as a start point time tₘ). The measurement unit 12 sets the time of the timing in the middle of the next stance phase at the timing of the start point time tₘ as the time of the end point of one walking cycle (also referred to as end point time tₘ₊₁).

In practice, the timing at which the roll angle becomes maximum/minimum does not completely coincide with the timing of the toe off/heel strike. Therefore, the walking waveform may be normalized in such a way that the timing at which the roll angle becomes maximum/minimum coincides with the timing of toe off/heel strike. For example, the measurement unit 12 normalizes the walking waveform in such a way that a section from the start point time tₘ to the time t_{b} is 30% of one walking cycle, a section from the time t_{b} to the time t_{d+1} is 40% of one walking cycle, and a section from the time t_{d+1} to the end point time tₘ₊₁ is 30% of one walking cycle. By normalizing the walking waveform, it is possible to align the timings of the appearance of different walking events depending on the person.

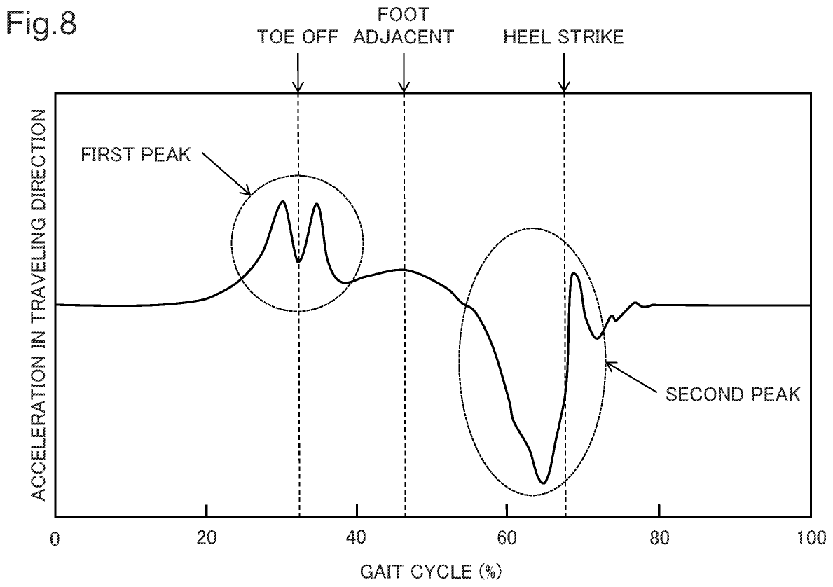

For example, the measurement unit 12 may detect the timing of the toe off/heel strike from the walking waveform of the acceleration in the traveling direction (acceleration in the Y direction). FIG. 8 is an example of the walking waveform measured by the measurement unit 12. FIG. 8 is an example of the walking waveform of the acceleration in the Y direction for one walking cycle starting from the timing in the middle of the stance phase (the start of the terminal stance period). In the walking waveform of the acceleration in the Y direction for one walking cycle, two main peaks (a first peak and a second peak) appear. The first peak appears around 20% to 40% of the walking cycle. The first peak includes two maximum peaks and one minimum peak. A timing of the minimum peak included in the first peak is the timing of the toe off. The second peak appears around 50% to 70% of the walking cycle. The second peak includes a minimum peak around a percentage of the walking cycle exceeding 60% and a maximum peak around 70% of the walking cycle. A timing in the midpoint between the minimum peak and the maximum peak included in the second peak is the timing of heel strike. A maximum timing of the gentle peak between the first peak and the second peak is the timing of foot adjacent. For example, the measurement unit 12 may detect, as the walking event, the tibia vertical or the foot adjacent, the heel rise, the opposite toe off, and the opposite heel strike. A method of detecting these walking events is omitted.

The measurement unit 12 calculates the gait parameter based on the detected walking event. For example, the measurement unit 12 calculates the gait parameter by using the timing of the detected walking event or the values of the sensor data at the timings of these walking events. For example, the measurement unit 12 calculates the gait parameter for each walking cycle. For example, the measurement unit 12 calculates the gait parameters such as a walking speed or a step length, a ground contact angle, a ground separation angle, a maximum foot lifting height (sensor position), circumduction (loci in the traveling direction), and a toe direction. A description of a method of calculating these gait parameters is omitted.

The measurement unit 12 transmits the gait parameter in the swing phase period in which the measurement of the sensor data is hardly affected. For example, the measurement unit 12 transmits the gait parameter for each step. For example, the measurement unit 12 may transmit the gait parameter for each walking cycle. For example, the measurement unit 12 transmits the gait parameter at intervals of seconds. The measurement unit 12 deletes the sensor data used to calculate the transmitted gait parameter from the buffer. The gait parameter transmitted from the measurement unit 12 is received by the mobile terminal (not illustrated) carried by the user. The measurement unit 12 may transmit the gait parameter via a wired line such as a cable or may transmit the gait parameter via wireless communication. For example, the measurement unit 12 is configured to transmit the gait parameter via a wireless communication function (not illustrated) conforming to a standard such as Bluetooth (registered trademark). The communication function of the measurement unit 12 may conform to a standard other than Bluetooth (registered trademark).

The mobile terminal (not illustrated) is a communication device that can be carried by a user. For example, the mobile terminal is a portable terminal device having a communication function, such as a smartphone, a smart watch, a tablet, or a mobile phone. The mobile terminal receives the gait parameter from the gait measurement device 10. For example, the mobile terminal executes data processing related to the body condition of the user by using the received gait parameter by application software or the like installed in the mobile terminal. For example, the mobile terminal causes a result of data processing of the gait parameter to be displayed on the screen of the mobile terminal. For example, the result of data processing of the gait parameter may be displayed on a screen of a terminal device (not illustrated) visually recognizable by the user. For example, the mobile terminal causes any numerical value of the gait parameter received from the measurement unit 12 to be displayed on the screen in real time. For example, the mobile terminal causes the time-series data of the gait parameter received from the measurement unit 12 to be displayed on the screen in real time. The mobile terminal may transmit the received gait parameter to a server, a cloud, or the like. The application of the gait parameter received by the mobile terminal is not particularly limited.

In a case in which the gait parameters are consecutively transmitted in real time, a communication period is set in a calculation period (also referred to as a gait data collection routine) of a series of gait parameters. For example, the communication period is set to a timing of the swing phase that hardly affects the measurement of the sensor data. Therefore, the communication after the measurement for one walking cycle is completed interrupts the gait data collection routine, and data is lost in the communication period. If the priority of the communication of the gait parameter is set to be high, the interruption of the sensor data measurement is stopped in the communication period, and thus, a sampling counter is also stopped at the same time. Due to the loss of the sensor data in the communication period, an error occurs in the gait parameter calculated using the sensor data.

In a case in which the gait measurement device 10 is implemented by a single-task microcomputer, the physical quantity detected by the sensor 11 is not acquired by the measurement unit 12 in the communication period of the gait parameter. Therefore, the physical quantity detected by the sensor 11 in the communication period of the gait parameter is not included in the sensor data measured by the measurement unit 12. That is, the sensor data measured by the measurement unit 12 has a loss of the communication period.

For example, when a dual-core microcomputer (also referred to as a multi-task microcomputer) is used, the measurement of the sensor data can be continued even during the communication period. The multi-task microcomputer has higher power consumption than the single-task microcomputer. In a case in which the gait measurement device 10 is mounted on an insole or the like of footwear, it is desirable that the power consumption of the gait measurement device 10 is as small as possible. Therefore, in the present example embodiment, an example using a single-task microcomputer is mainly used. Even in a case in which a multi-task microcomputer is used, the measurement of the sensor data may be stopped in the communication period depending on allocation of processing to the core. Therefore, the technique of the present example embodiment may be applied not only to the single-task microcomputer but also to the multi-task microcomputer.

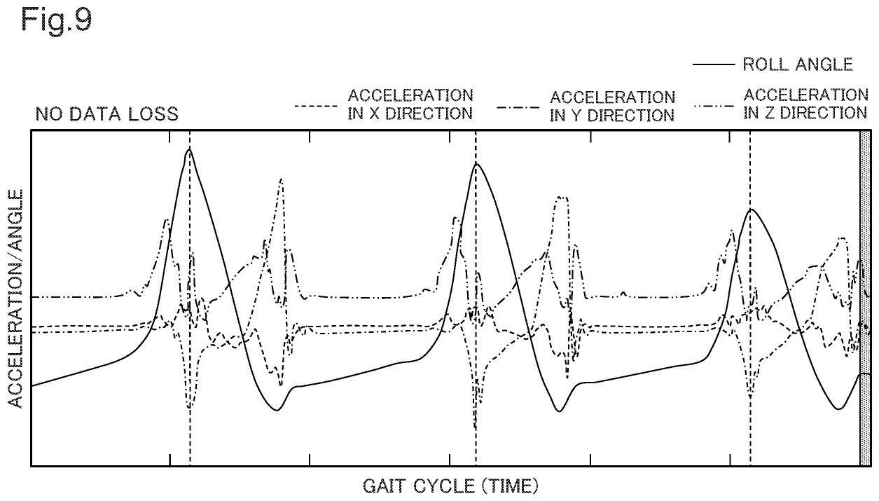

FIG. 9 is an example of the walking waveform in a case in which there is no data loss. FIG. 9 illustrates the walking waveform of the roll angle (solid line), the acceleration in the X direction (broken line), the acceleration in the Y direction (alternate long and short dash line), and the acceleration in the Z direction (alternate long and two short dashes line) for three walking cycles. In FIG. 9, the timing of the walking cycle at which the roll angle becomes maximum is indicated by a dotted line segment.

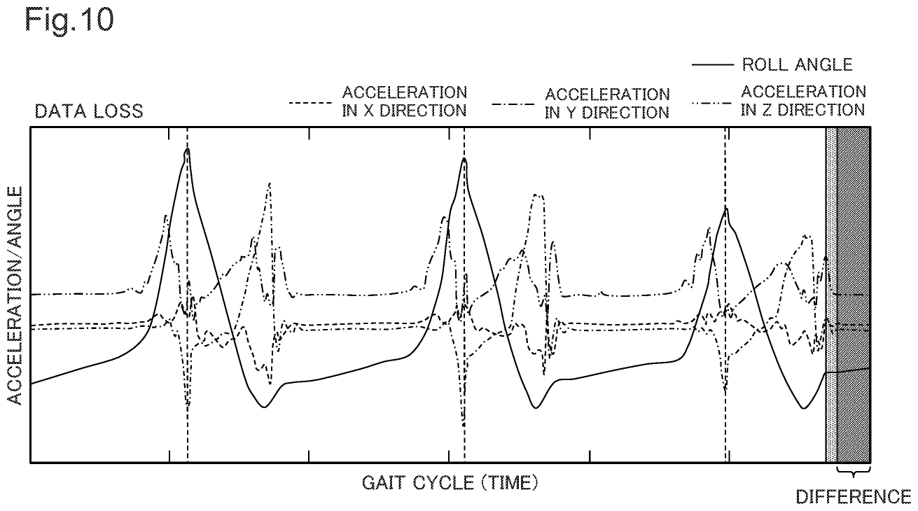

FIG. 10 is an example of the walking waveform in a case in which there is data loss. In the walking waveform of FIG. 10, the sensor data is lost in the communication period included in the swing phase. Therefore, as compared with the walking waveform of FIG. 9, in the walking waveform of FIG. 10, the walking cycle (dotted line) in which the roll angle becomes maximum shifts to the left along with walking. In addition, in the walking waveform of FIG. 10, since the loss of data for three walking cycles is accumulated, a difference from the walking waveform of FIG. 9 occurs at the end of the three walking cycles.

For example, in a case in which the gait measurement device 10 is mounted on both the left and right feet, when a defective portion over several meters is continuously connected, an error is likely to increase between the right foot and the left foot. For example, even if the loss time is the same, a difference in walking between the right and left feet is reflected, and an error of 5 to 10 centimeters (cm) may occur for each step when it is converted to a length. When such an error occurs, it is difficult to accurately measure the walking speed and the stride length for each step.

For example, walking of a patient during rehabilitation or a person with frailty tends to fluctuate at all times. In averaged data, it is often difficult to sufficiently grasp the condition of a patient who is undergoing rehabilitation or a person with frailty. Therefore, in the case of evaluating the state of rehabilitation or frailty, it is necessary to measure an accurate gait using consecutively measured sensor data. In the case of determining rehabilitation or frailty, the gait parameter is obtained based on the sensor data for each step instead of the averaged sensor data, and thus it is desirable to interpolate a section in which the data loss has occurred.

The measurement unit 12 interpolates the loss of the sensor data in the communication period or the like. Assuming that the gait parameters are continuously transmitted in real time, it is desirable that the process of interpolating the loss of the sensor data is as simple as possible. For example, the measurement unit 12 performs linear interpolation on the defective portion of the sensor data in the communication period.

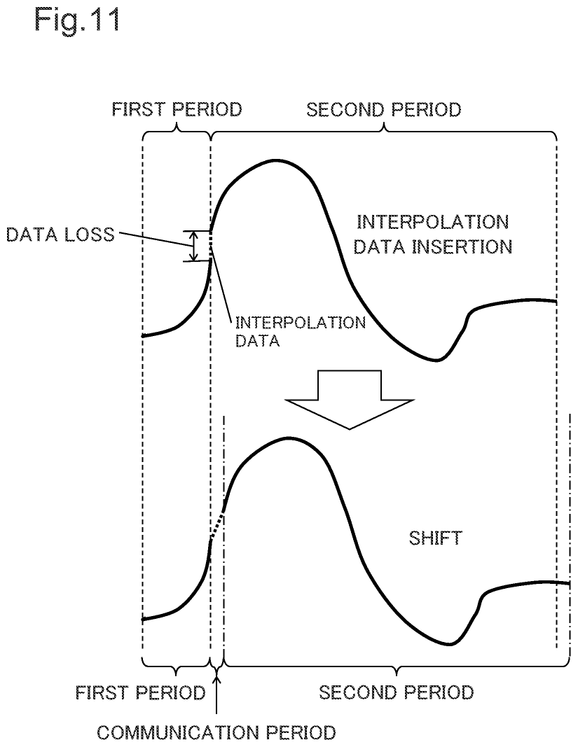

FIG. 11 is a conceptual diagram for describing an example of interpolating the loss of the sensor data by the measurement unit 12. In the example of FIG. 11, a discontinuous portion (data loss) occurs between a first period before the loss of the sensor data occurs and a second period after the loss of the sensor data occurs. First, the measurement unit 12 performs linear interpolation on a position at which the data loss has occurred. That is, the measurement unit 12 inserts interpolation data as long as the communication period between the end point of the first period and the start point of the second period. Next, the measurement unit 12 shifts the sensor data of the second period to the side (right side) with the larger walking cycle by the length identical to the communication period. At this time, the measurement unit 12 shifts the sensor data of the second period so that the interpolation data linearly connects a portion between the end point of the first period and the start point of the second period. As a result, the sensor data in which the interpolation data as long as the communication period is linearly interpolated between the end point of the first period and the start point of the second period is obtained. For example, the measurement unit 12 may insert the communication period between the end point of the first period and the start point of the second period, and then perform the linear interpolation on the interpolation data between the end point of the first period and the start point of the second period.

For example, the measurement unit 12 may offset the defective portion of the sensor data in the communication period with the sensor data before and after the defective portion. In other words, the measurement unit 12 may interpolate the data loss in the communication period by using either the data before or after the defective portion of the sensor data in the communication period. For example, the measurement unit 12 inserts the sensor data measured at measurement timings before and after the communication period between the end point of the first period and the start point of the second period by the number of points of the communication period. For example, the measurement unit 12 shifts the sensor data of the second period by the number of points of the communication period in the direction (right direction) in which the walking cycle increases, and inserts the value of the sensor data at the end point of the first period between the first period and the second period. For example, the measurement unit 12 inserts the value of the sensor data at the start point of the second period between the first period and the second period. For example, the measurement unit 12 inserts an average value such as an arithmetic mean value or a geometric mean value of the sensor data at the end point of the first period and the start point of the second period between the first period and the second period.

In a case in which the gait measurement device 10 and the mobile terminal (not illustrated) are constantly connected by wireless communication, the amount of data to be transmitted is substantially constant, and thus the communication period is substantially constant. For example, in a case in which the communication period is 40 milliseconds (ms) and the communication interruption is 10 ms, the data loss in the communication period includes four points. For example, in a case in which the sensor data (9-axis data) of the accelerations in the three axial directions, the angular velocities around the three axes, and the three axes are measured, 36(=4×9) pieces of data are interpolated.

It is desirable that the communication period of the gait parameter is not set in a period in which the stride determination is affected, such as the maximum/minimum roll angle or the vicinity of the heel strike/toe off. That is, the communication period of the gait parameter is preferably set in a period in which the gait parameter is hardly affected. For example, the communication period of the gait parameter is set in the period of the swing phase. For example, the communication period of the gait parameter is set to the start point of the swing phase (immediately after the toe off). In a case in which communication is started at the start point of the swing phase, interpolation data may be inserted after the start point of the swing phase, and the sampling counter may be simultaneously counted up. For example, the communication period may be set in a section in which the time-series data of the sensor data monotonically increases/monotonically decreases. In a case in which the communication period is set in a section in which the time-series data of the sensor data monotonically increases/monotonically decreases, linear interpolation is easily performed.

For example, the transmission timing of the gait data is set to the timing at which the swing phase starts. In a case in which the stride determination is performed in response to detection of the heel strike or the mid-stance period after the walking is detected, the section (period of time) of the stance phase and the swing phase is found. For example, the communication period may be set by using, as a marker, setting of a flag of the start of the swing phase (toe off). It is desirable that the communication period is set by using a timing after a little period of time elapses from the toe off as a starting point. The communication period may be a section between the toe off and the heel strike (swing phase), but since the feature related to walking is included, it is desirable to avoid a timing at which the roll angle shows the maximum.

For example, the communication period may be set in a period in which the entire sole contacts in the stance phase. For example, the communication period is set in a period in which the entire sole contacts from the heel strike to the heel rise. However, in a period in which the entire sole contacts, it may be difficult for the mobile terminal (not illustrated) to receive a wireless signal. Therefore, the communication period is preferably set in the swing phase rather than the stance phase.

### [Detailed Configuration]

Next, detailed configurations of the sensor 11 and the measurement unit 12 included in the gait measurement device 10 will be described with reference to the drawings. The following description will proceed with an example in which the sensor 11 includes an acceleration sensor and an angular velocity sensor.

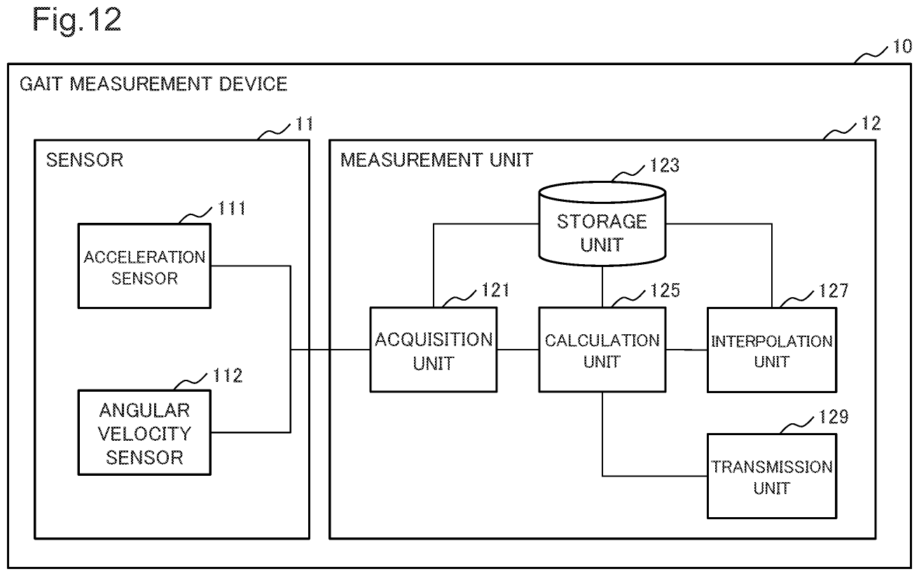

FIG. 12 is a block diagram for describing detailed configurations of the sensor 11 and the measurement unit 12. The sensor 11 includes an acceleration sensor 111 and an angular velocity sensor 112. The sensor 11 includes a power source (not illustrated). The measurement unit 12 includes an acquisition unit 121, a storage unit 123, a calculation unit 125, an interpolation unit 127, and a transmission unit 129.

The acceleration sensor 111 is a sensor that measures the accelerations (also referred to as spatial accelerations) in the three axial directions. The acceleration sensor 111 outputs the measured accelerations to the measurement unit 12. For example, a sensor of a piezoelectric type, a piezoresistive type, a capacitance type, or the like can be used as the acceleration sensor 111. The sensor used as the acceleration sensor 111 is not limited to the measurement method as long as the sensor can measure the acceleration.

The angular velocity sensor 112 is a sensor that measures the angular velocities (also referred to as spatial angular velocities) in the three axial directions. The angular velocity sensor 112 outputs the measured angular velocities to the measurement unit 12. For example, a sensor of a vibration type, a capacitance type, or the like can be used as the angular velocity sensor 112. The sensor used as the angular velocity sensor 112 is not limited to the measurement method as long as the sensor can measure the angular velocity.

When activated, the acquisition unit 121 operates in a vibration detection mode. For example, the acquisition unit 121 is activated in response to the user's operation and operates in the vibration detection mode. For example, the acquisition unit 121 is activated at a preset timing and operates in the vibration detection mode. In the vibration detection mode, the acquisition unit 121 acquires the sensor data from the sensor 11, and detects vibration derived from the walking in accordance with the value of the sensor data. For example, when the value of the sensor data exceeds a predetermined reference value, the acquisition unit 121 shifts to the measurement mode. When shifting to the measurement mode, the acquisition unit 121 samples the sensor data at a specified sampling rate. The measurement mode includes a measurement period, a gait parameter calculation period, and a communication period.

In the measurement period, the acquisition unit 121 acquires the accelerations in the three axial directions and the angular velocity around the three axes from each of the acceleration sensor 111 and the angular velocity sensor 112. The acquisition unit 121 converts the acquired accelerations and angular velocities into digital data, and stores the converted digital data (also referred to as sensor data) in the storage unit 123. The acquisition unit 121 may be configured to directly output the sensor data to the calculation unit 125. The sensor data includes at least acceleration data converted into digital data and angular velocity data converted into digital data. The acceleration data includes acceleration vectors in the three axial directions. The angular velocity data includes angular velocity vectors around the three axes. Acquisition times of the acceleration data and the angular velocity data are associated with the acceleration data and the angular velocity data. The acquisition unit 121 may add correction such as a mounting error, temperature correction, or linearity correction to the acquired acceleration data and angular velocity data. The acquisition unit 121 may generate angle data around the three axes by using the acquired acceleration data and angular velocity data. In the present example embodiment, the accelerations in the three axial directions and the angular velocities around the three axes are also referred to as sensor data.

The storage unit 123 stores the sensor data acquired by the acquisition unit 121. The sensor data stored in the storage unit 123 is used for the calculation of the gait parameter by the calculation unit 125. The sensor data stored in the storage unit 123 is used for data interpolation by the interpolation unit 127.

The calculation unit 125 acquires the sensor data from the storage unit 123 in the gait parameter calculation period. The calculation unit 125 may be configured to directly acquire the sensor data from the acquisition unit 121. In a stage in which the sensor data includes a defect, the calculation unit 125 acquires the sensor data which has undergone the data interpolation performed by the interpolation unit 127. The sensor data after the second walking cycle (second step) includes data loss as long as the communication period.

For example, the calculation unit 125 converts the coordinate system of the acquired sensor data from the local coordinate system to the world coordinate system. In a state in which the user is standing upright, the local coordinate system (the x axis, the y axis, and the z axis) and the world coordinate system (the X axis, the Y axis, and the Z axis) coincide with each other. While the user is walking, since a spatial posture of the sensor 11 changes, the local coordinate system (the x axis, the y axis, and the z axis) and the world coordinate system (the X axis, the Y axis, and the Z axis) do not coincide with each other. Therefore, the calculation unit 125 converts the sensor data acquired by the sensor 11 from the local coordinate system (the x axis, the y axis, and the z axis) of the sensor 11 into the world coordinate system (the X axis, the Y axis, and the Z axis). In a case in which the walking event can be detected using the sensor data of the local coordinate system, the coordinate conversion from the local coordinate system to the world coordinate system may be omitted.

By using the sensor data, the calculation unit 125 generates the time-series data of the physical quantity related to the motion of the foot measured along with walking of the pedestrian wearing the footwear on which the sensor 11 is installed. For example, the calculation unit 125 generates the time-series data such as the spatial acceleration or the spatial angular velocity. The calculation unit 125 performs integral on the spatial acceleration and the spatial angular velocity, and generates the time-series data such as the spatial velocity, the spatial angle (sole angle), or the spatial loci. These time-series data items are associated with the walking waveform. The calculation unit 125 generates the time-series data at a predetermined timing or at time intervals set in accordance with a general walking cycle or a walking cycle unique to the user. A timing at which the calculation unit 125 generates the time-series data may be set to any timing. For example, the calculation unit 125 is configured to continuously generate the time-series data during a period in which walking of the user is continued. The calculation unit 125 may be configured to generate the time-series data at a specific timing.

The calculation unit 125 extracts the time-series data (also referred to as the walking waveform) of the sensor data of one walking cycle from the generated time-series data. For example, the calculation unit 125 detects the timing in the middle of the stance phase as the start point of the walking waveform as the start point of the time-series data. For example, the calculation unit 125 may detect the timing of the heel strike or the toe off as the start point of the walking waveform.

The calculation unit 125 detects the walking event from the extracted walking waveform of one walking cycle. For example, the calculation unit 125 detects the walking events such as the heel strike, the toe off, the foot adjacent, the heel rise, the tibia vertical, the opposite toe off, and the opposite heel strike. The calculation unit 125 calculates the gait parameter based on the detected walking event. For example, the calculation unit 125 calculates the gait parameters such as a walking speed or a step length, a ground contact angle, a ground separation angle, a maximum foot lifting height (sensor position), circumduction (loci in the traveling direction), and a toe direction.

The interpolation unit 127 interpolates the data loss in the communication period. The description of the data interpolation by the measurement unit 12 can be applied to the data interpolation by the interpolation unit 127. For example, the interpolation unit 127 stores the sensor data which has undergone the data interpolation in the storage unit 123. For example, the interpolation unit 127 may output the sensor data which has undergone the data interpolation to the calculation unit 125.

The transmission unit 129 acquires the sensor data from the measurement unit 12. The transmission unit 129 transmits the acquired sensor data to the mobile terminal (not illustrated). For example, the transmission unit 129 transmits the sensor data to the mobile terminal via a wired line such as a cable. For example, the transmission unit 129 transmits the sensor data to the mobile terminal via wireless communication. For example, the transmission unit 129 is configured to transmit the sensor data to the mobile terminal via a wireless communication function (not illustrated) conforming to a standard such as Bluetooth (registered trademark) or WiFi (registered trademark). The communication function of the transmission unit 129 may conform to a standard other than Bluetooth (registered trademark) or WiFi (registered trademark).

### (Operation)

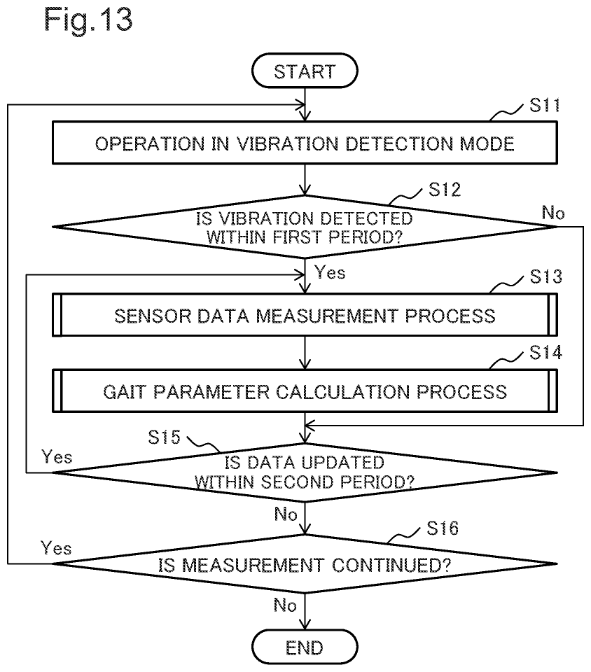

Next, an example of an operation of the gait measurement device 10 will be described with reference to the drawings. FIG. 13 is a flowchart for explaining an example of the operation of the gait measurement device 10. In the description of the process according to the flowchart of FIG. 13, the measurement unit 12 of the gait measurement device 10 is set as a main operation entity.

In FIG. 13, first, the measurement unit 12 operates in the vibration detection mode (step S11). For example, the measurement unit 12 is activated in response to the user's operation and operates in the vibration detection mode. For example, the measurement unit 12 is set to be activated in a preset time zone or timing.

When the vibration is detected within the first period during the operation in the vibration detection mode (Yes in step S12), the measurement unit 12 executes the sensor data measurement process (step S13). The first period is a period in which the measurement unit 12 operates in the vibration detection mode after being activated. The first period is set in advance. For example, the measurement unit 12 detects the vibration derived from walking in accordance with the value of the sensor data. In the sensor data measurement process of step S13, the measurement unit 12 measures the sensor data (step S13). Details of the sensor data measurement process of step S13 will be described later. When the vibration is not detected within the first period (No in step S12), the process proceeds to step S15.

After the sensor data measurement process of step S13, the measurement unit 12 executes the gait parameter calculation process (step S14). In the gait parameter calculation process of step S14, the measurement unit 12 calculates the gait parameter by using the sensor data measured in the sensor data measurement process of step S13. Details of the gait parameter calculation process of step S14 will be described later.

After step S14 or in the case of No in step S12, when there is data update within the second period (Yes in step S15), the process returns to step S13. The second period is a period in which the sensor data is continuously measured after the vibration is detected. The second period is set in advance. When there is no data update within the second period (No in step S15), the process proceeds to step S16.

When the measurement is continued (Yes in step S16), the process returns to step S11. When the measurement is not continued (No in step S16), the process according to the flowchart of FIG. 13 is ended. The continuation/stop of the measurement may be determined at a predetermined timing, in response to a stop operation of the user, or the like.

### [Sensor Data Measurement Process]

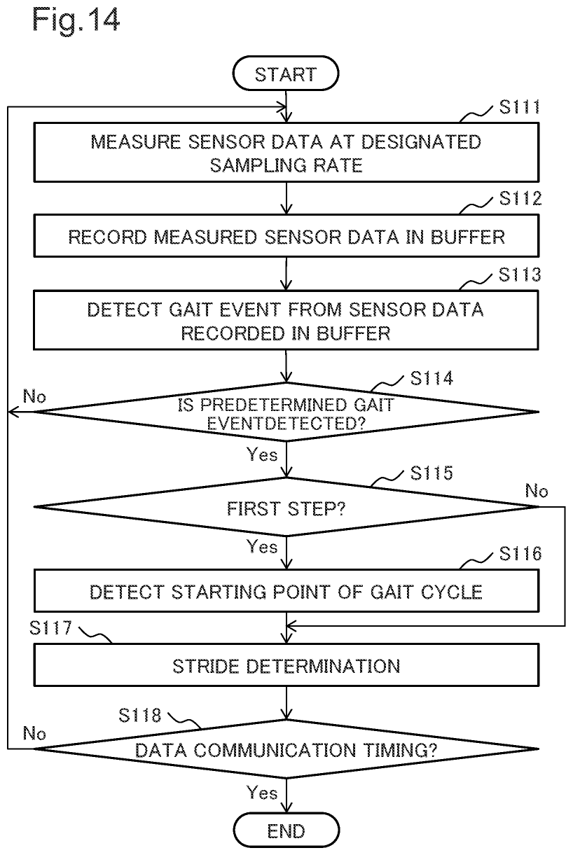

Next, an example of the sensor data measurement process (step S13 of FIG. 13) by the gait measurement device 10 will be described with reference to the drawings. FIG. 14 is a flowchart for describing an example of the sensor data measurement process by the gait measurement device 10. In the description of the process according to the flowchart of FIG. 14, the measurement unit 12 of the gait measurement device 10 is set as a main operation entity.

In FIG. 14, first, the measurement unit 12 measures the sensor data at a designated sampling rate (step S111). The measurement unit 12 acquires the sensor data such as the acceleration or the angular velocity from the sensor 11.

Next, the measurement unit 12 records the acquired sensor data in the buffer (the storage unit 123) (step S112).

Next, the measurement unit 12 detects the walking event from the sensor data recorded in the buffer (step S113). For the sensor data of the second step or later, the data loss in the communication period is interpolated.

When the predetermined walking event is detected (Yes in step S114) and this is the first step (Yes in step S115), the measurement unit 12 detects the start point of the walking cycle (step S116). For example, the measurement unit 12 detects the heel strike, the toe off, the timing in the middle of the stance phase, and the like as the start point of the walking cycle. When it is not the first step (No in step S115), the process proceeds to step S117.

After step S116 or in the case of No in step S115, the measurement unit 12 performs the stride determination (step S117). In the stride determination, the measurement unit 12 determines that the sensor data of one step (of one stride) has been acquired.

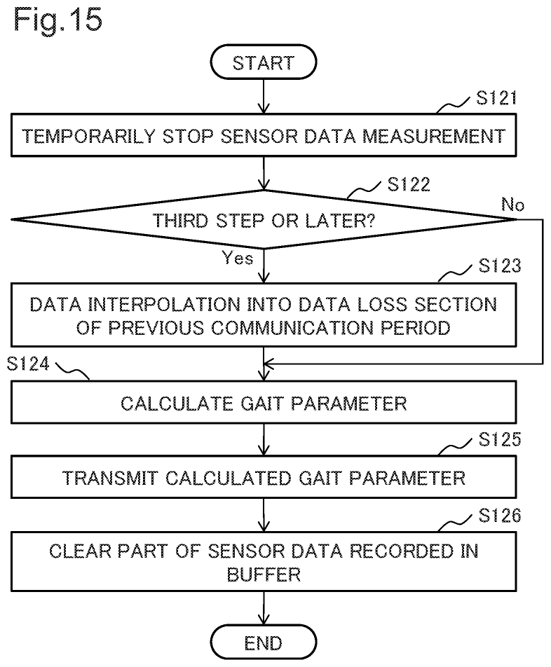

Here, when it is the timing of the data communication (Yes in step S118), the process proceeds to step S14 of the flowchart in FIG. 15 (step S121 of FIG. 13). For example, the timing of the data communication is the timing at which the swing phase starts. For example, the timing of the data communication is set by using a timing after a little period of time has elapsed from the toe off as the start point. For example, the timing of the data communication is set in a period avoiding the timing at which the roll angle becomes maximum in the swing phase. When it is not the timing of the data communication (No in step S118), the process returns to step S111.

### [Gait Parameter Calculation Process]

Next, an example of the gait parameter calculation process (step S14 of FIG. 13) by the gait measurement device 10 will be described with reference to the drawings. FIG. 15 is a flowchart for describing an example of the gait parameter calculation process by the gait measurement device 10. In the description of the process according to the flowchart of FIG. 15, the measurement unit 12 of the gait measurement device 10 is set as a main operation entity.

In FIG. 15, first, the measurement unit 12 temporarily stops the measurement of the sensor data (step S121). In the case of the single-task microcomputer, it is difficult to perform the sensor data measurement and the gait parameter communication at the same time, and thus the sensor data measurement is temporarily stopped.

In the case of the third step or later (Yes in step S122), the measurement unit 12 performs data interpolation on the data loss of the previous communication period (step S123). The third step here is the first step of the second walking cycle after walking is detected. The sensor data which has undergone the data interpolation is stored in the buffer (the storage unit 123). If it is before the third step (No in step S122), the process proceeds to step S124.

After step S123 or in the case of No in step S122, the measurement unit 12 calculates the gait parameter by using the sensor data stored in the buffer (the storage unit 123) (step S124). In the case of the first step, since there is no data loss, the measurement unit 12 calculates the gait parameter by using the measured sensor data. In the case of the second step or later, since there is a data loss, the measurement unit 12 calculates the gait parameter by using the sensor data which has undergone the data interpolation. For example, the measurement unit 12 calculates the gait parameters such as a walking speed or a step length, a ground contact angle, a ground separation angle, a maximum foot lifting height (sensor position), circumduction (loci in the traveling direction), and a toe direction.

Next, the measurement unit 12 transmits the calculated gait parameter (step S125). For example, the measurement unit 12 transmits the gait parameters such as a walking speed or a step length, a ground contact angle, a ground separation angle, a maximum foot lifting height (sensor position), circumduction (loci in the traveling direction), and a toe direction.

Next, the measurement unit 12 clears a part of the sensor data stored in the buffer (the storage unit 123) (step S126). For example, the measurement unit 12 deletes the sensor data used to calculate the transmitted gait parameter from the buffer (the storage unit 123). After step S126, the process proceeds to step S15 of the flowchart of FIG. 13.

As described above, the gait measurement device of the present example embodiment includes the sensor and the measurement unit. The sensor includes the acceleration sensor that measures accelerations in the three axial directions and the angular velocity sensor that measures the angular velocities around the three axes. The sensor outputs the sensor data measured by the acceleration sensor and the angular velocity sensor to the measurement unit. The measurement unit includes the acquisition unit, the calculation unit, the interpolation unit, and the transmission unit. The acquisition unit acquires the sensor data related to the motion of the foot. The interpolation unit interpolates the interpolation data into a period in which the sensor data is lost. The calculation unit calculates the gait parameter by using the sensor data obtained by interpolating the interpolation data by the interpolation unit. The transmission unit transmits the gait parameter calculated by the calculation unit.

The gait measurement device of the present example embodiment interpolates the interpolation data in the period in which the sensor data is lost, and calculates the gait parameter by using the sensor data obtained by interpolating the interpolation data. Therefore, according to the gait measurement device of the present example embodiment, it is possible to interpolate the loss of the sensor data and perform the highly accurate gait measurement.

In an aspect of the present example embodiment, the acquisition unit stops the acquisition of the sensor data during the communication period of the gait parameter by the transmission unit. The interpolation unit interpolates the loss of the sensor data in the communication period. According to the present aspect, the loss of the sensor data in the communication period of the gait parameter is interpolated, and thus the highly accurate gait measurement can be performed.

In one aspect of the present example embodiment, the interpolation unit performs the linear interpolation between the sensor data acquired immediately before and immediately after the communication period. According to the present aspect, the loss of the sensor data can be interpolated by performing the linear interpolation on the interpolation data during the communication period.

In one aspect of the present example embodiment, the interpolation unit interpolates the loss of the sensor data in the communication period by using the sensor data acquired immediately before or immediately after the communication period. According to the present aspect, the loss of the sensor data can be interpolated by using the sensor data acquired immediately before or immediately after the communication period.

In one aspect of the present example embodiment, the interpolation unit interpolates the sensor data acquired immediately before or immediately after the communication period as the sensor data in the communication period. According to the present aspect, the loss of the sensor data can be interpolated by inserting the sensor data acquired immediately before or after the communication period into the communication period.

In one aspect of the present example embodiment, the interpolation unit interpolates the average value of the sensor data acquired immediately before and immediately after the communication period as the sensor data in the communication period. According to the present aspect, the loss of the sensor data can be interpolated by inserting the average value of the sensor data acquired immediately before and after the communication period into the communication period.

In a case in which the gait measurement device of the present example embodiment is installed in the insole or the like of the footwear of the user, the data measured by the gait measurement device is transmitted to the mobile terminal of the user or the like by wireless communication such as Bluetooth (registered trademark). In this case, for example, the single-task microcomputer having small power consumption is used as hardware for implementing the gait measurement device. In order to reduce the power consumption in the communication, it is required to reduce the communication opportunity of the gait parameter and to reduce the capacity of the data transmitted to the mobile terminal as much as possible. For example, the communication opportunities of the gait parameters can be reduced by measuring the gait parameters of several steps and transmitting the average value of the gait parameters of several steps. In a case in which the single-task microcomputer is used, it is difficult to calculate the gait parameter by using the sensor data in the communication period of the gait parameter. In a case in which the gait parameter based on the sensor data is verified in real time, when the gait parameter is calculated in a state in which the sensor data of the communication period is lost, the influence of the loss of the sensor data becomes more significant as the number of steps increases. As a result, the accuracy of the gait parameter decreases.

According to the technique of the present example embodiment, it is possible to measure the gait parameter with high accuracy by interpolating the loss of the sensor data in the communication period for each step. By using the technique of the present example embodiment, even in a case in which the gait parameter based on the sensor data is verified in real time, since the data loss in the communication period is interpolated, the gait parameter can be measured with high accuracy.

### Second Example Embodiment

Next, a gait measurement system according to a second example embodiment will be described with reference to the drawings. The gait measurement system of the present example embodiment includes the gait measurement device of the first example embodiment. The gait measurement system of the present example embodiment executes the data processing related to the body condition of the user by using the gait parameter measured by the gait measurement device.

### (Configuration)

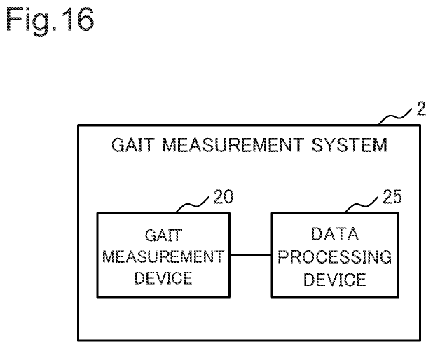

FIG. 16 is a block diagram illustrating an example of a configuration of the gait measurement system 2 according to the present example embodiment. The gait measurement system 2 includes a gait measurement device 20 and a data processing device 25.

The gait measurement device 20 has a configuration similar to the gait measurement device 10 of the first example embodiment. The gait measurement device 20 is installed on the user's footwear. When the vibration is detected within the first period during the operation in the vibration detection mode, the gait measurement device 20 executes the sensor data measurement process. The gait measurement device 20 calculates the gait parameter by using the measured sensor data. For the third step or later (the second walking cycle or later), the gait measurement device 20 calculates the gait parameter by using the sensor data which has undergone the data interpolation. The gait measurement device 20 transmits the calculated gait parameter to the data processing device 25.

For example, the gait measurement device 20 transmits the gait parameter at the timing of the swing phase. For example, the gait measurement device 20 transmits the gait parameter for each step. For example, the gait measurement device 20 may transmit the gait parameter for each walking cycle. The gait measurement device 20 deletes the sensor data used to calculate the transmitted gait parameter from the buffer.

The gait parameter transmitted from the gait measurement device 20 is received by the mobile terminal (not illustrated) carried by the user. The gait measurement device 20 may transmit the gait parameter via a wired line such as a cable or may transmit the gait parameter via wireless communication. For example, the gait measurement device 20 is configured to transmit the gait parameter via a wireless communication function (not illustrated) conforming to a standard such as Bluetooth (registered trademark). The communication function of the gait measurement device 20 may conform to a standard other than Bluetooth (registered trademark).

The mobile terminal (not illustrated) is a communication device that can be carried by a user. For example, the mobile terminal is a portable communication device having a communication function, such as a smartphone, a smart watch, or a mobile phone. The mobile terminal receives the gait parameter from the gait measurement device 20. For example, the mobile terminal processes the received gait parameter by the data processing device 25 installed in the mobile terminal. For example, the mobile terminal transmits the received gait parameter to the data processing device 25 installed in a server (not illustrated) or a cloud (not illustrated). In the present example embodiment, it is assumed that the data processing device 25 is installed in the mobile terminal.

The data processing device 25 acquires the gait parameter from the gait measurement device 20. The data processing device 25 executes the data processing related to the body condition in accordance with the gait of the user by using the gait parameter acquired from the gait measurement device 20. For example, the data processing device 25 determines the symmetry of walking of the user by using the gait parameter. For example, the data processing device 25 estimates the degree of progression of the hallux valgus of the user using the gait parameter. For example, the data processing device 25 performs personal identification of the user or personal authentication of the user using the gait parameter. For example, the data processing device 25 calculates the step length or the stride length of the user by using the gait parameter. For example, the data processing device 25 estimates the degree of pronation/supination of the user by using the gait parameter. For example, the data processing device 25 performs measurement related to the lower limb of the user by using the gait parameter. The data processing by the data processing device 25 is not limited to the example described herein as long as the gait parameter acquired from the gait measurement device 20 is used. A specific method of the data processing by the data processing device 25 will not be described.

The data processing device 25 outputs a result of the data processing on the gait parameter. For example, the data processing device 25 causes the result of the data processing on the gait parameter to be displayed on the screen of the mobile terminal in which the data processing device 25 is installed. For example, the data processing device 25 causes any numerical value of the gait parameter received from the gait measurement device 20 to be displayed on the screen of the mobile terminal in real time. For example, the data processing device 25 causes the time-series data of the gait parameter received from the gait measurement device 20 to be displayed on the screen of the mobile terminal in real time. For example, the data processing device 25 causes information related to the body condition of the user estimated by using the gait parameter received from the gait measurement device 20 or information associated with the estimated body condition to be displayed on the screen of the mobile terminal. For example, the data processing device 25 may transmit the received gait parameter to a server, a cloud, or the like. The application of the gait parameter received by the mobile terminal is not particularly limited.

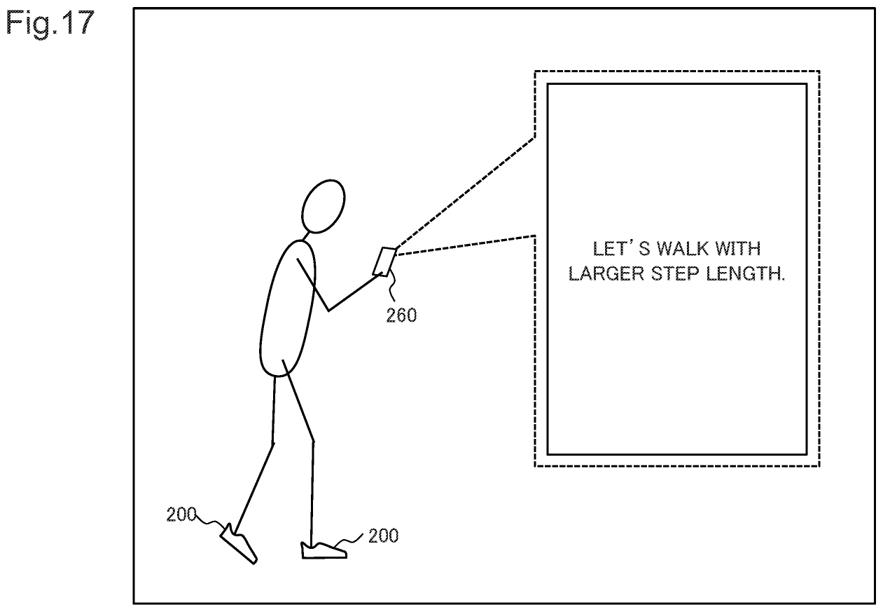

FIG. 17 is an example in which information associated with walking of the user is displayed on a screen of a mobile terminal 260 carried by the user walking in shoes 200 on which the gait measurement device 20 is installed. In the example of FIG. 17, recommendation information associated with the body condition of the user estimated by using the gait parameter received from the gait measurement device 20 is displayed on the screen of the mobile terminal 260. In the example of FIG. 17, recommendation information such as “Let's walk with larger step length” is displayed on the screen of the mobile terminal 260 in association with the body condition of the user estimated using the gait parameter (step length). The user who has checked the recommendation information displayed on the screen of the mobile terminal 260 may be able to improve his/her health condition by improving walking in accordance with the recommendation information.

For example, the data processing device 25 estimates a symptom of the foot of the user or the degree of recovery from injury in accordance with the degree of variation in the left and right step lengths of the user. For example, in a case in which the degree of variation in the left and right step lengths is larger than before, there is a possibility that the symptom is progressing or the injury is getting worse. In this case, there is a possibility that the symptom or injury of the user can be improved by causing information recommending the medical examination in the hospital to be displayed on the screen of the mobile terminal 260 of the user. For example, when the degree of variation in the left and right step lengths is smaller than before, there is a possibility that the user tends to recover from symptoms or injuries. In this case, when information indicating the trend of recovery is displayed on the screen of the mobile terminal 260 of the user, motivation of the user for rehabilitation or the like is likely to be improved.

For example, in a case in which influence of a sprain or an old wound of the foot affects motion of the ankle, the influence is reflected on a value of the ground contact angle/ground separation angle or lateral balance. Therefore, it is possible to verify the degree or state of recovery of a sprain or an old wound depending on the magnitude of the value of the ground contact angle/ground separation angle or the lateral balance. For example, in a case in which the value of the ground contact angle/ground separation angle of the foot having the sprain or an old wound is less than a predetermined value, there is a possibility that the symptom of the user can be improved by causing information recommending an examination or a treatment to be displayed on a screen of the mobile terminal 260 of the user. For example, in a case in which the value of the ground contact angle/ground separation angle of the foot having a sprain or an old wound exceeds a predetermined value, there is a possibility that the quality of life of the user is improved by causing information indicating the trend of recovery to be displayed on the screen of the mobile terminal 260 of the user.

For example, when a foot lifting height associated with an absolute value of clearance decreases, the risk of stumbling or falling over due to a step or the like increases. Therefore, the risk of falling can be verified by verifying the foot lifting height. For example, in a case in which the foot lifting height falls below a predetermined value, there is a possibility that the fall risk of the user can be avoided by causing information recommending an examination, a treatment, or training to be displayed on the screen of the mobile terminal 260 of the user. For example, in a case in which the foot lifting height exceeds a predetermined value, there is a possibility that the quality of life of the user is improved by causing information indicating that the user is in a healthy walking state to be displayed on the screen of the mobile terminal 260 of the user.

For example, in a situation in which a patient is visiting the hospital for rehabilitation of leg symptoms or injuries, the patient walks in front of a doctor, and the doctor determines the state of the leg. However, there may be a case in which an aspect different from daily walking is exhibited in front of the doctor, depending on the psychological state of the user. Therefore, it is desirable that the body condition can be determined based on a numerical value or an index measured in daily life. Since the gait measurement system of the present example embodiment can measure/estimate a numerical value or an index indicating the state of the foot in daily life, it is easy to make accurate determination without being affected by the psychological state of the user. In addition, since the gait measurement system of the present example embodiment can detect the state of the user in real time in daily life, even in a case in which a symptom or a medical condition rapidly deteriorates, the gait measurement system can respond flexibly by making emergency contact with a hospital or the like.

As described above, the gait measurement system of the present example embodiment includes the gait measurement device and the data processing device. The gait measurement device includes the acceleration sensor that measures the accelerations in the three axial directions and the angular velocity sensor that measures the angular velocities around the three axes. The gait measurement device calculates the gait parameter by using the sensor data measured by the acceleration sensor and the angular velocity sensor. The gait measurement device interpolates the interpolation data into the period in which the sensor data is lost. The gait measurement device calculates the gait parameter by using the sensor data obtained by interpolating the interpolation data. The gait measurement device transmits the calculated gait parameter to the data processing device. The data processing device acquires the gait parameter transmitted by the gait measurement device installed in the foot part of the user. The data processing device executes the data processing related to the body condition of the user by using the gait parameter.

The gait measurement system of the present example embodiment interpolates the interpolation data into the period in which the sensor data is lost, and calculates the gait parameter by using the sensor data obtained by interpolating the interpolation data. Therefore, according to the gait measurement system of the present example embodiment, it is possible to interpolate the loss of the sensor data and perform the highly accurate gait measurement.

In an aspect of the present example embodiment, the data processing device causes the information related to the body condition of the user obtained by the data processing using the gait parameter to be displayed on the screen of the terminal device visually recognizable by the user. According to the present aspect, the user can check the body condition of the user displayed on the screen of the terminal device.

### Third Example Embodiment

Next, a gait measurement device according to a third example embodiment will be described with reference to the drawings. The gait measurement device of the present example embodiment has a configuration in which a sensor is omitted from a first gait measurement device. The gait measurement device of the present example embodiment has a configuration in which a measurement unit of the first gait measurement device is simplified.

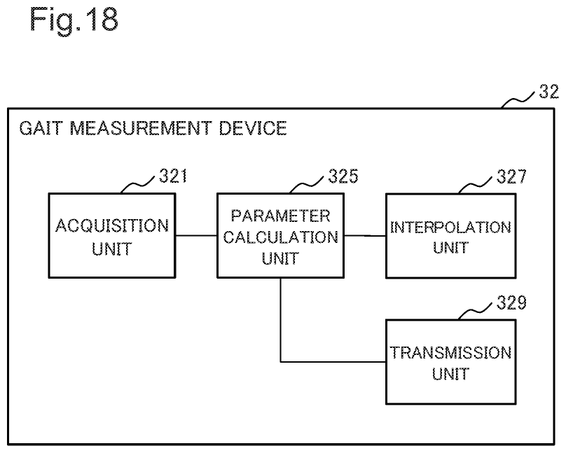

FIG. 18 is a block diagram illustrating an example of a configuration of a measurement device 32 of the present example embodiment. The measurement device 32 includes an acquisition unit 321, a calculation unit 325, an interpolation unit 327, and a transmission unit 329.

The acquisition unit 321 acquires the sensor data related to the motion of the foot. The interpolation unit 327 interpolates the interpolation data into a period in which the sensor data is lost. The calculation unit 325 calculates the gait parameter by using the sensor data interpolated with the interpolation data by the interpolation unit 327. The transmission unit 329 transmits the gait parameter calculated by the calculation unit 325.

The gait measurement device of the present example embodiment interpolates the interpolation data in the period in which the sensor data is lost, and calculates the gait parameter by using the sensor data obtained by interpolating the interpolation data. Therefore, according to the gait measurement device of the present example embodiment, it is possible to interpolate the loss of the sensor data and perform the highly accurate gait measurement.

### (Hardware)

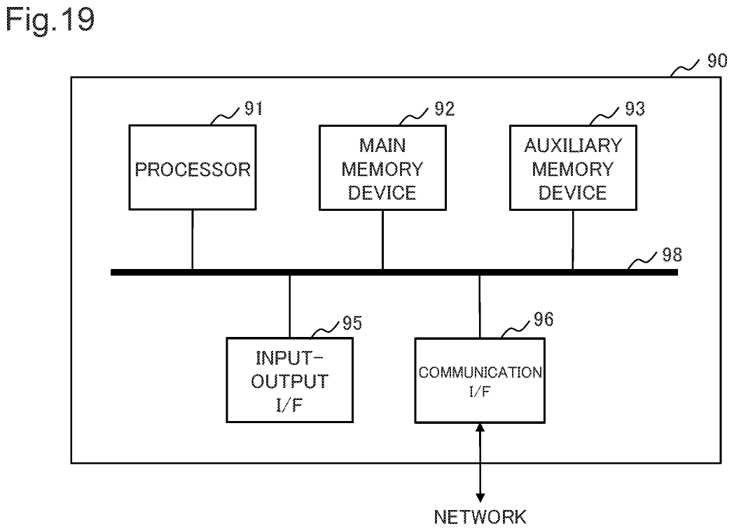

Here, a hardware configuration for executing control or processing according to each example embodiment of the present disclosure will be described with an example of an information processing device 90 of FIG. 19. The information processing device 90 on FIG. 19 is a configuration example for executing control or processes of each example embodiment and not intended to limit the scope of the present disclosure.

As illustrated in FIG. 19, the information processing device 90 includes a processor 91, a main memory device 92, an auxiliary memory device 93, an input-output interface 95, and a communication interface 96. In FIG. 19, the interface is abbreviated as I/F. The processor 91, the main memory device 92, the auxiliary memory device 93, the input-output interface 95, and the communication interface 96 are connected to one another via a bus 98 so that data communication can be performed. The processor 91, the main memory device 92, the auxiliary memory device 93, and the input-output interface 95 are connected to a network such as the Internet or an intranet via the communication interface 96.

The processor 91 causes a program stored in the auxiliary memory device 93 or the like to be developed in the main memory device 92. The processor 91 executes the program developed in the main memory device 92. In the present example embodiment, a software program installed in the information processing device 90 may be used. The processor 91 executes control or processing according to each example embodiment.

The main memory device 92 has an area in which a program is developed. A program stored in the auxiliary memory device 93 or the like is developed in the main memory device 92 by the processor 91. The main memory device 92 is implemented by, for example, a volatile memory such as a dynamic random access memory (DRAM). A non-volatile memory such as a magnetoresistive random access memory (MRAM) may be configured/added as the main memory device 92.

The auxiliary memory device 93 stores various pieces of data such as programs. The auxiliary memory device 93 is implemented by a local disk such as a hard disk or a flash memory. In a case in which various pieces of data are stored in the main memory device 92, the auxiliary memory device 93 may be omitted.

The input-output interface 95 is an interface for connecting the information processing device 90 with peripheral devices based on a standard or a specification. The communication interface 96 is an interface for connecting to external systems or devices via a network such as the Internet or an intranet based on a standard or a specification. The input-output interface 95 and the communication interface 96 may be combined as an interface that provides connection with external devices.

Input devices such as a keyboard, a mouse, and a touch panel may be connected to the information processing device 90 as necessary. These input devices are used to input information or settings. In a case in which the touch panel is used as the input device, the display screen of the display device may also serve as the interface of the input device. Data communication between the processor 91 and the input device may be mediated by the input-output interface 95.

The information processing device 90 may be provided with a display device for displaying information. In a case in which the display device is provided, it is desirable that the information processing device 90 includes a display control device (not illustrated) for controlling display of the display device. The display device is connected to the information processing device 90 via the input-output interface 95.

The information processing device 90 may be provided with a drive device. The drive device mediates reading of data or a program from a recording medium (program recording medium), writing of a processing result of the information processing device 90 in the recording medium, or the like between the processor 91 and the recording medium. The drive device is connected to the information processing device 90 via the input-output interface 95.

The example of the hardware configuration for enabling control or processes according to each example embodiment of the present invention has been described above. The hardware configuration of FIG. 19 is an example of the hardware configuration for executing control or processes according to each example embodiment and not intended to limit the scope of the present invention. A program for causing a computer to execute control or processing according to each example embodiment is also included in the scope of the present invention. A program recording medium in which the program according to each example embodiment is recorded is also included in the scope of the present invention. The recording medium can be implemented by, for example, an optical recording medium such as a compact disc (CD) or a digital versatile disc (DVD). The recording medium may be implemented by a semiconductor recording medium such as a universal serial bus (USB) memory or a secure digital (SD) card. Furthermore, the recording medium may be implemented by a magnetic recording medium such as a flexible disk or other recording media. In a case in which the program executed by the processor is recorded in the recording medium, the recording medium is substantially equivalent to the program recording medium.

The components of each example embodiment may be combined in any form. The components of each example embodiment may be implemented by software or may be implemented by a circuit.

Although the present invention has been described with reference to the example embodiments, the present invention is not limited to the above example embodiments. Various modifications that can be understood by those skilled in the art can be made to the configuration or details of the present invention within the scope of the present invention.

Some or all of the above example embodiments may be described as the following supplementary notes, but the present invention is not limited to the following supplementary notes.

### (Supplementary Note 1)

A gait measurement device, comprising:
an acquisition unit that acquires sensor data related to a motion of a foot;
an interpolation unit that interpolates interpolation data into a period in which the sensor data is lost;
a calculation unit that calculates a gait parameter by using the sensor data obtained by interpolating the interpolation data by the interpolation unit; and
a transmission unit that transmits the gait parameter calculated by the calculation unit.

### (Supplementary Note 2)

The gait measurement device according to supplementary note 1, wherein
the acquisition unit stops the acquisition of the sensor data in a communication period of the gait parameter by the transmission unit, and
the interpolation unit interpolates loss of the sensor data in the communication period.

### (Supplementary Note 3)

The gait measurement device according to supplementary note 2, wherein
the interpolation unit performs linear interpolation between the sensor data acquired immediately before and immediately after the communication period.

### (Supplementary Note 4)

The gait measurement device according to supplementary note 2, wherein
the interpolation unit interpolates the loss of the sensor data in the communication period by using the sensor data acquired immediately before or immediately after the communication period.

### (Supplementary Note 5)

The gait measurement device according to supplementary note 4, wherein
the interpolation unit inserts the sensor data acquired immediately before or immediately after the communication period as the sensor data in the communication period.

### (Supplementary Note 6)

The gait measurement device according to supplementary note 4, wherein
the interpolation unit inserts an average value of the sensor data acquired immediately before and immediately after the communication period as the sensor data in the communication period.

### (Supplementary Note 7)

The gait measurement device according to any one of supplementary notes 1 to 6, further comprising a sensor that includes an acceleration sensor that measures accelerations in three axial directions and an angular velocity sensor that measures angular velocities around three axes, and outputs the sensor data measured by the acceleration sensor and the angular velocity sensor to the acquisition unit.

### (Supplementary Note 8)

A gait measurement system, comprising:
the gait measurement device according to any one of supplementary notes 1 to 7; and
a data processing device that acquires the gait parameter transmitted by the gait measurement device installed in a foot portion of a user, and executes data processing related to a body condition of the user by using the gait parameter.

### (Supplementary Note 9)

The gait measurement system according to claim 8, wherein
the data processing device causes information related to the body condition of the user obtained by the data processing using the gait parameter to be displayed on a screen of a terminal device visually recognizable by the user.

### (Supplementary Note 10)

A gait measurement method performed by a computer, comprising:
acquiring sensor data regarding a motion of a foot;
interpolating interpolation data into a period in which the sensor data is lost;
calculating a gait parameter by using the sensor data obtained by interpolating the interpolation data; and
transmitting the calculated gait parameter.

### (Supplementary Note 11)

A program causing a computer to execute:
a process of acquiring sensor data regarding a motion of a foot;
a process of interpolating interpolation data into a period in which the sensor data is lost;
a process of calculating a gait parameter by using the sensor data obtained by interpolating the interpolation data; and
a process of transmitting the calculated gait parameter.

### REFERENCE SIGNS LIST

2 gait measurement system
10, 20 gait measurement device
11 sensor
12 measurement unit
25 data processing device
111 acceleration sensor
112 angular velocity sensor
121, 321 acquisition unit
123 storage unit
125, 325 calculation unit
127, 327 interpolation unit
129, 329 transmission unit

What is claimed is:

1. A gait measurement device, comprising:
a first memory storing instructions; and
a first processor connected to the first memory and configured to execute the instructions to:
acquire sensor data related to a motion of a foot;
interpolate interpolation data into a period in which the sensor data is lost;
calculate a gait parameter by using the sensor data obtained by interpolating the interpolation data; and
transmit the gait parameter.

2. The gait measurement device according to claim 1, wherein
the first processor is configured to execute the instructions to
stop the acquisition of the sensor data in a communication period of the gait parameter, and
interpolate loss of the sensor data in the communication period.

3. The gait measurement device according to claim 2, wherein
the first processor is configured to execute the instructions to
perform linear interpolation between the sensor data acquired immediately before and immediately after the communication period.

4. The gait measurement device according to claim 2, wherein
the first processor is configured to execute the instructions to
interpolate the loss of the sensor data in the communication period by using the sensor data acquired immediately before or immediately after the communication period.

5. The gait measurement device according to claim 4, wherein
the first processor is configured to execute the instructions to
insert the sensor data acquired immediately before or immediately after the communication period as the sensor data in the communication period.

6. The gait measurement device according to claim 4, wherein
the first processor is configured to execute the instructions to
insert an average value of the sensor data acquired immediately before and immediately after the communication period as the sensor data in the communication period.

7. The gait measurement device according to claim 1, further comprising
a sensor that includes an acceleration sensor that measures accelerations in three axial directions and an angular velocity sensor that measures angular velocities around three axes.

8. A gait measurement system, comprising:
the gait measurement device according to claim 1; and
a data processing device comprising
a second memory storing instructions; and
a second processor connected to the second memory and configured to execute the instructions to:
acquire the gait parameter transmitted by the gait measurement device installed in a foot portion of a user, and
execute data processing related to a body condition of the user by using the gait parameter.

9. The gait measurement system according to claim 8, wherein
the data processing device of the second processor is configured to execute the instructions to
cause information related to the body condition of the user obtained by the data processing using the gait parameter to be displayed on a screen of a terminal device visually recognizable by the user.

10. A gait measurement method performed by a computer, comprising:
acquiring sensor data regarding a motion of a foot;
interpolating interpolation data into a period in which the sensor data is lost;
calculating a gait parameter by using the sensor data obtained by interpolating the interpolation data; and
transmitting the calculated gait parameter.

11. A non-transitory recording medium storing a program causing a computer to execute:
a process of acquiring sensor data related to a motion of a foot;
a process of interpolating interpolation data into a period in which the sensor data is lost;
a process of calculating a gait parameter by using the sensor data obtained by interpolating the interpolation data; and
a process of transmitting the calculated gait parameter.
